(19) United States
(12) Patent Application Publication
(10) Pub. No.: US 2003/0236430 A1
(43) Pub. Date: Dec. 25, 2003

(54) Preparation of protected amino acids

(76) Inventors: David Robert Allen, La Grange Park, IL (US); Seung-Hee Kyung-Lee, Buffalo Grove, IL (US)

Correspondence Address: Kevin R. Erdman, Baker & Daniels, Suite 2700, 300 North Meridian Street, Indianapolis, IN, 46204, US

(21) Appl. No.: 10/177,174

(22) Filed: Jun. 21, 2002

(51) Int. Cl. C07C 229/24

(52) U.S. Cl. 560/171

(57) ABSTRACT

The present invention involves a process for preparing protected amino acids. The process produces a di-tert-butyl amino ester or an N-protected di-tert-butyl amino ester by transesterification of an acidic amino acid or an N-protected acidic amino acid. By-products of the transesterification reaction may be recycled for use as part of the starting material. The N-protected di-tert-butyl amino ester may be hydrogenated to form a di-tert-butyl amino ester, which may subsequently form a di-tert-butyl ester hydrocholride salt.

### FIELD OF THE INVENTION

This invention relates to a process for preparing amino acids having at least one carboxyl group protected by a tert-butyl group. Specifically, the process involves preparing a protected di-tert butyl amino ester from acidic amino acids.

### BACKGROUND OF THE INVENTION

It has been found that certain amino acid derivatives and several naturally occurring peptides have biological activities that may be used in diverse fields. Some are useful as antibiotics, growth factors, or smooth muscle stimulants. Synthesis of biological active peptides involves incorporation of desired amino acids into a peptide chain. Current methods for peptide synthesis, however, can be time consuming and can frequently give poor yields.

Amino acids are the basic structural unit of proteins. An α-amino acid consists of an amino group, a carboxyl group, a hydrogen atom, and a distinctive R group. The R group bonds to an α-carbon atom adjacent to the carboxyl (acidic) group. In certain instances, the R group may be referred to as a side chain. All proteins in all species, from bacteria to humans, are constructed from the same set of twenty amino acids, two of which contain acidic (R group) side chains. The two acidic amino acids are aspartic acid and glutamic acid.

From a reactivity stand point, amino acids contain a plurality of functional groups. These functional groups may be selected as points on the compound to facilitate the modification of the compound. When one of the functional groups is selected for chemical modification, the other functional groups may require protecting to prevent a production of unwanted by-products. Numerous protective groups are already known for being suitable for protecting various functional groups. One important property that is required of such protective groups is that they be able to be removed under mild conditions having the least possible effect on other protective groups or functional groups. Examples of protective groups which meet the requirement include the tert-butyl group, which is commonly used for protecting hydroxyl and carboxyl groups. The disadvantage of using the tert-butyl group is the complicated multi-step process of introduction to the functional group and the resulting low yields.

There is a need for simple processes for preparing protected amino acids that efficiently utilize the starting material and result in high yields.

### SUMMARY OF THE INVENTION

The present invention provides a process for preparing protected amino acids having at least one carboxyl group protected with a tert-butyl group. Specifically, the present invention involves a process for preparing a protected di-tert-butyl amino ester from an acidic amino acid. The di-tert-butyl amino ester has each of two carboxyl groups protected with a tert-butyl group.

In one embodiment of the present invention, the process for preparing a protected amino acid comprises the steps of providing an acidic amino acid or derivatives thereof, and subjecting the acidic amino acid or the derivatives to a transesterification reaction in the presence of a tert-butyl compound and a suitable catalyst. As a result, a di-tert-butyl amino ester is produced. The di-tert-butyl amino ester may be N-protected if an N-protected acidic amino acid is used as the starting material.

The acidic amino acid that may be used as the starting material may include aspartic acid (Asp), glutamic acid (Glu) and their derivatives. The derivatives may include N-protected Asp and N-protected Glu. Benzyloxycarbonyl-L-aspartic acid (Z-L-Asp) and benzyloxycarbonyl-L-glutamic acid (Z-L-Glu) are examples of the suitable starting material. The product of the transesterification reaction may include Z-aspartic acid-di-tert-butyl ester or Z-glutamic acid-di-tert-butyl ester.

The suitable catalyst comprises at least one of boron trifluoride complexes, sulfuric acid, methanesulfonic acid, zinc chloride, and titanium tetrachloride. Some examples of the boron trifluoride complexes include boron trifluoride diethyl etherate, boron trifluoride dibutyl etherate, boron trifluoride tert-butyl methyl etherate, boron trifluoride dimethyl etherate, boron trifluoride tetrahydrofuran, and boron trifluoride acetic acid.

The tert-butyl compound comprises tert-butyl acetate, tert-butyl benzoate, tert-butyl methacrylate, tert-butyl proprionate, and tert-butyl bromoacetate.

The transesterification reaction may further produce by-products including mono-tert-butyl esters. If Z-L-Asp is used as the starting material, the mono-tert-butyl esters include Z-L-Asp-α-tert-butyl ester and Z-L-Asp-β-tert-butyl ester. If Z-L-Glu is used as the starting material, mono-tert-butyl esters include Z-L-Glu-α-tert-butyl ester and Z-L-Glu-γ-tert-butyl ester. The by-products of the transesterification reaction may be recycled back into the transesterification reaction, above mentioned.

The process of the present invention may further comprise the step of hydrogenating the N-protected di-tert-butyl amino ester in the presence of a catalyst to form a di-tert-butyl amino ester, and the step of reacting the di-tert-butyl amino ester with a second acid to form a di-tert-butyl amino ester salt.

The above and other embodiments, aspects, alternatives and advantages of the present invention will become more apparent from the following detailed description of the present invention taken in conjunction with the examples.

### DETAILED DESCRIPTION OF THE INVENTION

For the purposes of promoting an understanding of the principles of the invention, specific language will be used to describe the embodiments of the invention. It will nevertheless be understood that no limitation of the scope of the invention is thereby intended. The invention includes any alterations and further modifications in the described products and methods and further applications of the principles of the invention which would normally occur to one skilled in the art to which the invention relates.

The present invention provides a novel process for preparing protected amino acids, particularly acidic amino acids. The process generally involves transesterification of an acidic amino acid in the presence of a tert-butyl compound and a suitable transesterification catalyst to produce a di-tert-butyl amino ester.

The acidic amino acid described herein may include both natural and unnatural amino acids that contain a terminal (α) carboxyl group and a side-chain (ω) carboxyl group. The natural acidic amino acids include aspartic acid and glutamic acid. The side-chain (ω) carboxyl group of aspartic acid is referred to as (β) carboxyl group, while the side-chain (ω) carboxyl group of glutamic acid is referred to as (γ) carboxyl group.

During the transesterification reaction, each carboxyl group of the acidic amino acid reacts with the tert-butyl compound to form a di-tert-butyl amino ester. The di-tert-butyl amino ester has both (α) and (ω) carboxyl groups protected by the butyl groups.

In addition to the di-tert-butyl amino ester, the transesterification reaction may produce mono-tert-butyl esters as by-products. The mono-tert-butyl esters contain only one tert-butyl group protecting either the (α) or the (ω) carboxyl group.

The acidic amino acid may further include the acidic amino acid derivatives such as N-protected acidic amino acids. For example, benzyloxycarbonyl-L-aspartic acid (Z-L-Asp) and benzyloxycarbonyl-L-glutamic acid (Z-L-Glu) are particularly suitable as the starting material.

According to the process of the present invention, Z-L-Asp or Z-L-Glu is mixed with a tert-butyl compound, which may be a solvent or prepared by dissolving in a solvent. The suitable tert-butyl compounds include tert-butyl acetate, tert-butyl benzoate, tert-butyl methacrylate, tert-butyl proprionate, and tert-butyl bromoacetate.

It has been found that any appropriate amount of the tert-butyl compound may be used. However, a mole ratio of 1 starting material to 10 tert-butyl compound works well.

To the reaction mixture, a suitable amount of a suitable catalyst is added. An example of the suitable catalyst is boron trifluoride diethyl etherate (BF₃.Et₂O). Other boron trifluoride complex such as boron trifluoride dibutyl etherate, boron trifluoride tert-butyl methyl etherate, boron trifluoride dimethyl etherate, boron trifluoride tetrahydrofuran, and boron trifluoride acetic acid may also be used. In addition, the suitable catalyst may include a first acid that is capable of acting as a catalyst. The first acid may include sulfuric acid and methanesulfonic acid. Certain salts such as zinc chloride and titanium tetrachloride also have been found to function as the suitable catalyst for the transesterification of an acidic amino acids and the derivatives thereof.

Generally, about 10 mole % of the catalyst, based on the mole amount of the starting material, is used. The optimal working range of the catalyst may be about 10 to about 30 mole %. Reducing the amount of the catalyst may slow down the reaction rate. However, the reaction will proceed with any amount of the catalyst.

The transesterification reaction may take place at room temperature or at an increased temperature. A suitable temperature may range from room temperature to about 50° C. It is suitable to stir the reaction mixture for at least 4.5 hours to 9 hours. In certain experiments using different catalysts, longer reaction time may be required.

Quenching by adding water to the reaction mixture may be necessary to stop the reaction. The pH of the quenched solution should be adjusted to about 10. Generally, it is suitable to use concentrated sodium hydroxide (10N NaOH) for the pH adjustment. While the pH is being adjusted, the temperature of the reaction mixture should be maintained at about 30° C.

After quenching, certain reaction mixtures may separate into three layers, a top layer, a middle layer, and a bottom layer. Generally, the top or organic layer contains di-tert-butyl amino ester, and the middle layer contains by-products including amino acid mono-tert-butyl esters. The top and the middle layers may be separately collected and processed to recover the di-tert-butyl amino ester or the amino acid mono-tert-butyl esters.

If Z-L-Asp is used as the starting material, the transesterification product is Z-Asp-di-tert-butyl ester (Z-Asp(OtBu)₂), and the by-products include Z-L-Asp-α-tert-butyl ester (Z-L-Asp-α-(OtBu)) and Z-L-Asp-tert-butyl ester (Z-L-Asp-β-(OtBu)).

If Z-L-Glu is used as the starting material, the transesterification product is Z-Glu-di-tert-butyl ester (Z-Glu(OtBu)₂), and the by-products include Z-L-Glu-α-tert-butyl ester (Z-L-Glu-α-(OtBu)), and Z-L-Glu-γ-tert-butyl ester (Z-L-Glu-γ-(OtBu)).

One benefit of the present invention is that large amounts of di-tert-butyl amino ester can be produced and easily isolated from the by-products of mono-tert-butyl esters.

Another benefit of the present invention is that the mono-tert-butyl esters may be recycled and used as part of the starting material for the above described transesterification.

In the cases in which the transesterification product is an N-protected di-tert-butyl amino ester, the next step of the process of the present invention may involve hydrogenating the N-protected di-tert-butyl amino ester to remove the N-protecting group to form a di-tert-butyl amino ester.

To initiate the hydrogenation reaction, the N-protected di-tert-butyl amino ester is dissolved in a solvent such as ethyl acetate to form a solution. The hydrogenation reaction may be run in the presence of a suitable catalyst such as palladium black, platinum, or other metals or metal-containing catalysts, under hydrogen pressure, and at room temperature. During the hydrogenation reaction, the N-protecting group such as the benzyloxycarbonyl group (Z) on the N-protected di-tert butyl amino ester is replaced with a hydrogen molecule. If Z-Asp(OtBu)₂ or Z-Glu(OtBu)₂ is the substrate for the hydrogenation reaction, the product is L-Asp-di-tert-butyl amino ester (L-Asp(OtBu)₂) or L-Glu-di-tert-butyl amino ester (L-Glu(OtBu)₂), respectively.

The following step of the process of the present invention involves a production of a di-tert-butyl amino ester salt. This salt formation step is accomplished by reacting the di-tert-butyl amino ester with a second acid, which may include hydrochloric acid (HCl), sulfuric acid, oxalic acid, phosphoric acid, and acetic acid. Other acids that are capable of forming a salt with the di-tert-butyl amino ester may also be used. The resulting salt may include L-Asp-di-tert-butyl amino ester hydrochloride salt (L-Asp(OtBu)₂.HCl) or L-Glu-di-tert-butyl amino ester hydrochloride salt (L-Glu(OtBu)₂.HCl).

The product of the present invention including the di-tert butyl amino esters or the di-tert butyl amino ester salts may be further processed by regioselective hydrolysis to produce amino acid (ω) mono-ester. The regioselective hydrolysis reaction may be facilitated by an enzyme such as an esterase or a lipase. For example, a pig liver esterase (PLE) may be used. The PLE enzyme is known to have an ability to selectively hydrolyze the (α) carboxyl group. The resulting compound is an (ω) mono-tert-butyl ester. Depending on the starting material used, the (ω) mono-tert-butyl ester may include L-Asp-β-tert-butyl ester (L-Asp-β-(OtBu)) or L-Glu-γ-tert-butyl ester (L-Glu-γ-(OtBu)).

After the (ω) mono-tert-butyl ester is produced, it may be further processed in a subsequent step involving adding a suitable N-protecting group, such as 9-fluorenylmethoxycarbonyl (Fmoc). The method for protecting the amino group with Fmoc is well known in the literature. An Fmoc group may be added to L-Asp-β-(OtBu) or L-Glu-γ-(OtBu) to form Fmoc-L-Asp(OtBu)-OH or Fmoc-L-Glu(OtBu)-OH, respectively. The resulting compound may be used in the production of pharmaceutical products.

The following non-limiting examples are presented to illustrate the invention which is not to be considered as limited thereto.

### EXAMPLE 1

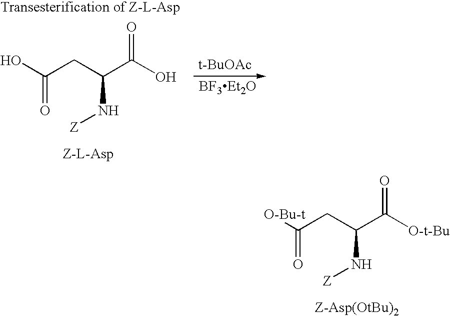

Thirty grams (0.112 mole) of Z-L Asp was mixed with 154 ml of tert-butyl acetate (t-BuOAc) to form a slurry. Then, 2.8 ml (0.0225 mol) of boron trifluoride diethyl etherate (BF₃.Et₂O) was added to the slurry. The slurry was stirred at room temperature for 8 hours 45 minutes. The slurry became a complete solution after about 3.5 hours. In-process UPLC (Guard Column: Whatman Partisil 5 ODS-3, Buffer: 4.2 mM NaH₂PO₄/10.8 mM Na₂HPO₄, pH=2.9, Run Time=32.00 min, Flow rate=1.000 ml/min, Oven Temperature=40° C., Injection volume=20.0 ml, Wavelength=210 nm, Solvent A=Buffer, Solvent B=Acetonitrile, Run Time=32.00 min, Flow rate=1.000 ml/min, Oven Temperature=40° C., Injection volume=20.0 ml, Wavelength=210 nm) was performed to determine the composition of the solution. The result indicated that the solution contained about 56.8 area % of Z-Asp di-tert-butyl ester (Z-Asp(OtBu)₂). The solution was quenched with 80 ml of water. The pH of the quenched solution was measured and was found to be 1.04, at a temperature of 23.1° C. The pH of the quenched solution was adjusted to a pH of 10 by adding 10 N NaOH (about 35 ml), while the temperature was maintained at less than 30° C. This process resulted in a final pH of 10.37 at 23.0° C. Three layers of solution were formed, a top layer, a middle layer and a bottom layer. The top or organic layer was collected and concentrated on a rotary evaporator (Rotovap, Brinkman Instruments, Westbury, N.Y.), at a temperature of about 45° C. to about 50° C. The resulting product had an appearance of a yellow oil. This product contained Z-Asp-di-tert-butyl ester, Z-Asp(OtBu)₂ The yield of Z-Asp(OtBu)₂ was assessed by HPLC to be about 55 to 60 area %.

### EXAMPLE 2

### Transesterification Using Different Catalyst

The transesterification reactions were set up in the same manner as described in EXAMPLE 1. However, in place of BF₃.Et₂O, an alternative catalyst was used. The catalysts tested were acids or salts, as listed in TABLE I below. Each reaction was run at room temperature or at about 50° C., for a period of about 5.5 to 15 hours. At the end of the reaction time, the reaction mixture was analyzed using the HPLC technique, as described in EXAMPLE 1. The results in TABLE I show that the transesterification reactions in the presence of certain catalysts, namely, concentrated sulfuric acid (H₂SO₄), methanesulfonic acid (MsOH), zinc chloride (ZnCl₂), and titanium tetrachloride in dichloromethane (TiCl4 in DCM), yielded significant amounts of the Z-Asp(OtBu)₂. In contrary, aluminum chloride (AlCl₃), ferric chloride (FeCl₃), and titanium tetraisopropoxide (Ti[OiPr]₄) were not as effective, even with the increase in the reaction temperature and the reaction time.

TABLE I
Results of transesterification reactions using different catalysts

| | | | Area % | | |
|---|---|---|---|---|---|
| | Time | | | | |
| Catalyst | (hrs) | Temp. | Z-L-Asp | Z-Asp(OtBu) | Z-Asp(OtBu)₂ |
| conc. | 5.5 | r.t. | 6.56 | 36.71 | 48.90 |
| H₂SO₄ | | | | | |
| HCl(4M in | 5.5 | r.t. | 88.29 | 2.30 | 0 |
| Dioxane) | | | | | |
| HCl(4M in | 15 | 50° C. | 85.88 | 5.67 | 0.17 |
| Dioxane) | | | | | |
| MsOH | 6.5 | r.t. | 5.96 | 36.27 | 48.55 |
| AlCl₃ | 5.5 | r.t. | 77.32 | 11.04 | 1.44 |
| AlCl₃ | 15 | 50° C. | 73.28 | 15.00 | 1.78 |
| FeCl₃ | 6.5 | r.t. | 85.96 | 3.66 | 0.18 |
| FeCl₃ | 15 | 50° C. | 79.04 | 9.16 | 0.45 |
| ZnCl₂ | 6.5 | r.t. | 89.50 | 0.13 | 0 |
| ZnCl₂ | 15 | 50° C. | 17.97 | 50.95 | 19.01 |
| Ti[OiPr]₄ | 6.5 | r.t. | 87.96 | 0 | 0 |
| Ti[OiPr]₄ | 15 | 50° C. | 77.43 | 1.03 | 0 |
| TiCl4(1 M | 5.5 | r.t. | 71.38 | 16.17 | 1.63 |
| in DCM) | | | | | |
| TiCl4(1 M | 15 | 50° C. | 54.74 | 33.14 | 4.09 |
| in DCM) | | | | | |

r.t. = room temperature

### EXAMPLE 3

### Transesterification Using Different tert-butyl Compound

The transesterification reactions were set up in the same manner described in EXAMPLE 1. However, in place of tert-butyl acetate, an alternative tert-butyl compound was added to the starting material (Z-L-Asp). The tert-butyl compounds tested were in the form of solvents. Each reaction was run at room temperature for about 4.5 to 5 hours. At the end of the reaction time, each mixture was analyzed using the UPLC technique, as described in EXAMPLE 1. The results in TABLE II show that the transesterification reactions in the presence of certain tert-butyl compounds, namely, tert-butyl benzoate, tert-butyl methacrylate, tert-butyl proprionate, and tert-butyl bromoacetate, yielded significant amounts of Z-Asp(OtBu)₂. In contrary, methyl-tert-butyl ether (MTBE), and tert-butyl formate were not as effective for producing Z-Asp(OtBu)₂ (TABLE II).

TABLE II
Results of transesterification reactions using different tert-
butyl compounds

| tert-butyl | Time | Area % | | |
|---|---|---|---|---|
| compound | (hrs) | Z-L-Asp | Z-Asp(OtBu) | Z-Asp(OtBu)₂ |
| methyl-tert-butyl | 4.5 | 76.56 | 19.68 | 0.99 |
| ether (MTBE) | | | | |
| methyl-tert-butyl | 48 | 65.76 | 27.25 | 2.15 |
| ether (MTBE) | | | | |
| tert-butyl- | 4.5 | 5.25 | 25.92 | 29.58 |
| bromoacetate | | | | |
| tert-butyl formate | 5 | 53.93 | 21.83 | 2.24 |
| tert-butyl | 5 | 0.96 | 17.55 | 68.56 |
| benzoate | | | | |
| tert-butyl | 5 | 3.14 | 22.78 | 45.76 |
| propionate | | | | |
| tert-butyl | 5 | 1.43 | 18.52 | 54.81 |
| methacrylate | | | | |

### EXAMPLE 4

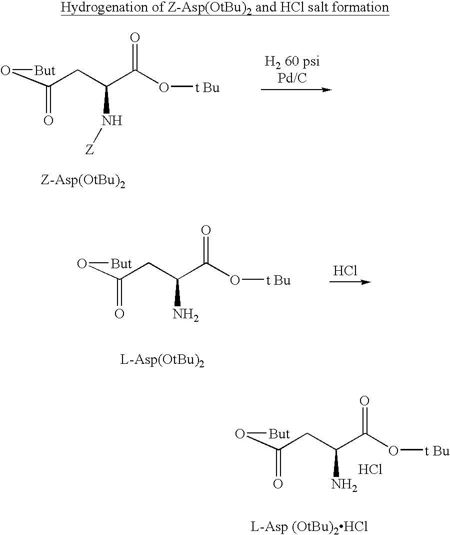

The oil of EXAMPLE 1 above was dissolved in 130 ml of ethyl acetate (EtOAc) and added to a Fisher Porter Bottle (Andrew Glass Co., Vineland, N.J.) containing 22 ml of 5% palladium on activated carbon (P/C), 61.81% water (1.99 g, 2.5 wt. %) and ethyl acetate. The resulting solution, which contained Z-Asp(OtBu)₂ was kept at room temperature, under a hydrogen pressure measured at 60 psi, for three hours. During this time, a hydrogenolysis reaction took place. Afterward, the solution was filtered over Celite® (Aldrich, Milwaukee, Wis.) to separate the metal-containing catalyst. The catalyst was rinsed twice with about 30 ml of ethyl acetate for reuse. The filtrate containing L-Asp di-tert-butyl amino ester (L-Asp(OtBu)₂) was cooled to a temperature of about 5° C. to 10° C. Then 18.9 ml (75.6 mmol) of 4M HCl (in dioxane) was added to the filtrate while stirring. The temperature of the filtrate-HCl reaction solution was maintained at a temperature of about 5° C. to 10° C. After stirring for about 30 minutes, the mixture was filtered. The wet cake was washed twice, each time with 25 ml of ethyl acetate. The washed wet cake was dried by suction for 30 minutes and then dried in vacuo at a temperature of about 50° C. A white solid obtained was L-Asp di-tert-butyl amino ester hydrochloride salt (L-Asp(OtBu)₂.HCl)(15.79 grams, 50% yield from Z-Asp.

While the invention has been illustrated and described in detail in the foregoing description, the same is to be considered as illustrative and not restrictive in character. It should be understood that only the exemplary embodiments have been described and that all changes and modifications that come within the spirit of the invention are desired to be protected.

What is claimed is:

1. A process for preparing a protected amino acid comprising the steps of:
providing an acidic amino acid; and
subjecting the acidic amino acid to a transesterification reaction in the presence of a tert-butyl compound and a suitable transesterification catalyst to produce a di-tert-butyl amino ester.

2. The process of claim 1, wherein the acidic amino acid comprises at least one of aspartic acid and glutamic acid.

3. The process of claim 1, wherein the acidic amino acid includes an acidic amino acid derivative.

4. The process of claim 3, wherein the acidic amino acid derivative includes N-protected acidic amino acid, and the di-tert-butyl amino ester includes an N-protected di-tert butyl amino ester.

5. The process of claim 4, wherein the N-protected acidic amino acid includes Z-L-aspartic acid and the N-protected di-tert butyl amino ester includes Z-L-aspartic acid di-tert-butyl amino ester.

6. The process of claim 4, wherein the N-protected acidic amino acid includes Z-L-glutamic acid and the N-protected di-tert butyl amino ester includes Z-L-glutamic acid di-tert-butyl amino ester.

7. The process of claim 1, wherein the tert-butyl compound comprises at least one of tert-butyl acetate, tert-butyl benzoate, tert-butyl methacrylate, tert-butyl proprionate, and tert-butyl bromoacetate.

8. The process of claim 1, wherein the transesterification catalyst comprises a boron trifluoride complex.

9. The process of claim 8, wherein the boron trifluoride complex includes at least one of boron trifluoride diethyl etherate, boron trifluoride dibutyl etherate, boron trifluoride tert-butyl methyl etherate, boron trifluoride dimethyl etherate, boron trifluoride tetrahydrofuran, and boron trifluoride acetic acid.

10. The process of claim 1, wherein the transesterification catalyst includes a first acid.

11. The process of claim 10, wherein the first acid includes at least one of sulfuric acid and methanesulfonic acid.

12. The process of claim 1, wherein the transesterification catalyst comprises a salt.

13. The process of claim 12, wherein the salt includes at least one of zinc chloride and titanium tetrachloride.

14. The process of claim 5, wherein the transesterification reaction further produces Z-L-Asp-α-tert-butyl ester and Z-L-Asp-β-tert-butyl ester as by-products.

15. The process of claim 14, wherein the by-products are recycled into the transesterification reaction of said subjecting step.

16. The process of claim 6, wherein the transesterification reaction further produces Z-L-Glu-α-tert-butyl ester and Z-L-Glu-γ-tert-butyl ester as by-products.

17. The process of claim 16, wherein the by-products are recycled into the transesterification reaction of said subjecting step.

18. The process of claim 4 further comprising the step of:
hydrogenating the N-protected di-tert-butyl amino ester in the presence of a suitable catalyst to produce a di-tert-butyl amino ester.

19. The process of claim 18, wherein the di-tert-butyl amino ester includes at least one of L-Asp-di-tert-butyl ester and L-Glu-di-tert-butyl ester.

20. The process of claim 18 further comprising the step of:
reacting the di-tert-butyl amino ester with a second acid to produce a di-tert-butyl amino ester salt.

21. The process of claim 20, wherein the second acid includes at least one of phosphoric acid, acetic acid, sulfuric acid, citric acid, and oxalic acid.

22. The process of claim 20, wherein the second acid includes hydrochloric acid and the di-tert-butyl amino ester salt includes di-tert-butyl amino ester hydrochloride salt.

23. The process of claim 22, wherein the di-tert-butyl amino ester hydrochloride salt includes at least one of L-Asp-di-tert-butyl amino ester hydrochloride salt and L-Glu-di-tert-butyl amino ester hydrochloride salt.

24. The process of claim 1 further comprising the step of:
reacting the di-tert-butyl amino ester with a second acid to produce a di-tert-butyl amino ester salt.

25. The process of claim 24, wherein the second acid includes at least one of phosphoric acid, acetic acid, sulfuric acid, and oxalic acid.

26. The process of claim 24, wherein the second acid includes hydrochloric acid and the di-tert-butyl amino ester salt includes di-tert-butyl amino ester hydrochloride salt.

27. The process of claim 26, wherein the di-tert-butyl amino ester hydrochloride salt includes at least one of L-Asp-di-tert-butyl amino ester hydrochloride salt and L-Glu-di-tert-butyl amino ester hydrochloride salt.
